(19) United States
(12) Patent Application Publication
(10) Pub. No.: US 2026/0103681 A1
(43) Pub. Date: Apr. 16, 2026

(54) AN ISOLATION AND CULTURE METHOD AND APPLICATION FOR PORCINE ENDOMETRIAL MESENCHYMAL STEM CELLS

(71) Applicant: Qingdao Kangmingbei Jian Biopharmaceutical Co., Ltd., Qingdao City (CN)

(72) Inventors: Wenhua XU, Qingdao City (CN); Xiao XU, Qingdao City (CN); Xiaomin WANG, Qingdao City (CN); Yan TANG, Qingdao City (CN); Quan TIAN, Qingdao City (CN); Xiaoying KONG, Qingdao City (CN); Liang ZHANG, Qingdao City (CN); Dan HAN, Qingdao City (CN)

(73) Assignee: Qingdao Kangmingbei Jian Biopharmaceutical Co., Ltd., Qingdao City, SD (CN)

(21) Appl. No.: 19/208,604

(22) Filed: May 15, 2025

(30) Foreign Application Priority Data
Oct. 11, 2024 (CN) 2024114147700

(51) Int. Cl. C12N 5/077

(52) U.S. Cl. CPC C12N 5/0653; C12N 2506/1392; C12N 2509/00

(57) ABSTRACT

The present invention relates to cell biology and regenerative medicine, specifically to a method for isolating and culturing porcine endometrial mesenchymal stem cells (PEMSCs) and their applications. PEMSCs are sourced from uterine tissue of pigs, providing a streamlined process yielding a substantial quantity of cells. Cultured PEMSCs exhibit CD34−, CD11b−, CD44+, and CD105+ phenotypic markers and can differentiate into osteocytes, adipocytes, and chondrocytes in vitro. The method employs type II collagenase to degrade extracellular matrix collagen and DNase I to break down DNA, reducing cell aggregation. This combination ensures efficient tissue digestion and high-quality single-cell suspensions. Applications include tissue repair for intrauterine adhesions, skin injuries, and vascular damage. The repair involves combining PEMSCs with biomaterials and implanting them into lesion sites. This method offers a simple and effective approach for cell transplantation therapy, enabling new possibilities in regenerative medicine.

### TECHNICAL FIELD

The present invention belongs to the field of cell biology and regenerative medicine technology, which specifically relates to an isolation and culture method and application for porcine endometrial mesenchymal stem cells.

### BACKGROUND OF THE INVENTION

The development of regenerative medicine and tissue engineering, which integrates theories and methods from multiple disciplines such as life sciences, engineering, and computer science, as well as modern biotechnological techniques like materials science, cell technology, and genetic engineering, has provided new means and methods for tissue regeneration and repair. These advancements enable the repair, replacement, and enhancement of damaged, diseased, or defective tissues and organs within the human body. A key component of regenerative medicine, stem cell therapy, has a wide range of applications in the medical field, with some product pipelines already entering the clinical stage. Stem cells, known for their potent differentiation and regeneration capabilities as well as their immunomodulatory effects, have become a hot topic in regenerative medicine research while also facing many challenges, including the selection of suitable cell sources, which has long troubled researchers

Mesenchymal stem cells (MSCs) are pluripotent stem cells with multi-directional differentiation potential and self-renewal ability, and have the potential to regenerate various tissues, organs and cells, also, the related clinical studies have been widely carried out and many major breakthroughs have been made. Endometrial MSCs (EMSCs) are cells located in the functional and basal layers of the endometrium, they are responsible for the regeneration and remodeling of the endometrium during the menstrual cycle, and they can differentiate into a variety of cell types, including endothelial cells, smooth muscle cells and osteoblasts. EMSCs have shown great potential in regenerative medicine, particularly in the treatment of endometrial injury and the promotion of endometrial regeneration. Studies have shown that EMSCs can promote angiogenesis and stromal cell proliferation by secreting growth factors or exosomes, and regulate inflammatory responses and inhibiting the regeneration of fibrotic endometrium at the same time. In the repair process of endometrial injury, EMSCs can migrate to the damaged site, and promote cell proliferation, angiogenesis, gland formation, regeneration of epithelial and stromal components, thereby improving the thickness and integrity of the endometrium. EMSCs also showed a regulatory effect on the inflammatory response, which can reduce the expression of pro-inflammatory factors and increase the expression of anti-inflammatory factors, which is helpful in creating an environment more conducive to embryo implantation. Additionally, EMSCs have also shown potential in the treatment of Asherman syndrome (AS). AS is a kind of endometrial adhesion caused by infection or trauma, resulting in infertility by reducing the available area for embryo implantation. As a local cell of the endometrium, EMSCs have natural advantages for periodic regeneration and have become an ideal cell source for repairing damaged endometrium. In general, EMSCs have significant potential in endometrial repair and regeneration, offering new strategies and hope for treating endometrial-related diseases and providing new therapeutic options for clinical applications.

### SUMMARY OF THE INVENTION

The specific process of the method for isolating and culturing PEMSCs (PEMSCs) involved in this invention includes the following steps:

### (1) Tissue Acquisition

Fresh porcine uterus (with ovaries) tissue is placed in cold phosphate-buffered saline (PBS) for preservation.

### (2) Tissue Pre-Treatment

Firstly, in the sterile room, cutting the tissue, using pre-cooled sterile PBS (pH=7.4) containing Ca²⁺ and Mg²⁺ at 4° C. for rinsing;

Secondly, the serosal layer (outermost layer) is removed, and the endometrial layer is separated from the myometrial layer;

Finally, the fragmented endometrial layer is digested in a basic culture medium supplemented with digestive enzymes at 37° C. for 2-3 hours in a culture incubator, with pipetting every 15 minutes to obtain a cell suspension.

### (3) Primary Centrifugation

Firstly, the cell suspension is filtered through 100- and 40-micron cell strainers. MSC complete culture medium (MSCCM) is added to stop further digestion. The cells are washed with PBS and centrifuged at 1000 rpm for 5 minutes;

Secondly, treating the cells with red cell lysis solution twice, followed by washing with PBS;

Finally, the cells are seeded at a clonal density of 500 cells/cm² in MSCCM and incubated at 37.0° C. in a humidified atmosphere with 5% CO₂. The day after cell seeding, the cell attachment status is closely observed, and the MSC complete culture medium is replaced every 2-3 days. These cells are referred to as passage 0 (P0) cells.

### (4) Culture and Passaging

Firstly, when the cell density reaches 60%-80%, the MSCCM, trypsin, and PBS are pre-warmed in a 37° C. water bath. Aspirate the existing MSCCM from the cell culture system, followed by washing the cells with PBS;

Secondly, 0.25% trypsin is added for digestion. When cell retraction and dispersion are observed under a microscope, an equal volume of complete culture medium is immediately added to stop the digestion. The cells are transferred to an EP tube and centrifuged at 1000 rpm for 5 minutes;

Finally, the supernatant is removed, and the cells are resuspended in MSCCM. One-third of the cells are transferred to a new culture dish, gently mixed, and cultured in a CO₂ incubator. The cell state is observed after 12 hours. These cells are referred to as passage 1 (P1) cells.

### (5) Cell Counting, Viability Testing, and Culture

Taking 100 μL of P1 generation cells, detecting the cell activity by trypan blue and counting, adjusting the cell density, inoculating the cells into a six-well plate containing MSCCM, placing in a cell incubator for cultivation, and the cells after subculture again being the P2 generation cells, and so on. The seeding density for passaged cells is 0.5×10⁴/cm²-1.5×10⁴/cm².

### (6) Induction of Differentiation

P2-P3 cells are taken and cultured in different cell differentiation induction media to induce differentiation osteoblasts, chondrocytes, adipocytes and so on by adding different cell differentiation mediums.

The concentration of magnesium chloride and calcium chloride added in sterile PBS in step (2) of the isolation and culture method for PEMSCs cells involved in this invention is 1 mM and 0.5 mM, respectively;

The digestive enzymes include 0.2% type II collagenase and 40 μg/mL deoxyribonuclease I (DNase I). The combination of type II collagenase and DNase I is used, where type II collagenase is responsible for digesting collagen in the extracellular matrix, and DNase I is responsible for degrading DNA to reduce cell aggregation, thereby promoting effective tissue digestion and obtaining high-quality single-cell suspensions. Most cells in the endometrium are endometrial cells, and the proportion of mesenchymal stem cells is relatively small. Type II collagenase hydrolyzes proline in the interstitial tissue to separate cells. It has strong digestive action on collagen and good digestive action on interstitial tissue, but it has little effect on the cells themselves, allowing epithelial cells to separate from collagen components without damage. DNase I can act on DNA released during cell separation, randomly cleaving DNA strands into oligodeoxynucleotides, reducing cell clumps, and helping to prepare single-cell suspensions.

In step (3), the antibiotic is a mixture containing 100 U/mL penicillin and 100 μg/mL streptomycin, commonly referred to as “double antibiotics”(DA).

The complete medium of MSC in steps (3) and (4) of the isolation and culture method for PEMSCs involved in this invention is MSC basal medium (MSCBM) containing 10% fetal bovine serum (FBS) and 1% DA.

The culture conditions of the cell culture incubator in step (5) are: temperature 37.0° C., 5% CO₂ saturation.

The induced differentiation of cells in the steps (6) of the isolation and culture method for PEMSCs involved in the present invention includes osteogenic differentiation, chondrogenic differentiation, adipogenic differentiation and other three-line induction and differentiation of MSCs, and the three-line induced differentiation (adipogenic induction, osteogenic induction, chondrogenic induction) are based on the steps of OriCell® adipose-derived MSCs osteogenic (GUXMD-90021), adipogenic (GUXMD-90031), chondrogenic (GUXMD-90041) induced differentiation kit to operate and verify the staining of calcium nodules, lipid droplets, and cartilage ball sections; wherein osteogenic differentiation medium induced eMSCs to differentiate into osteocytes; chondrogenic differentiation medium induced eMSCs to differentiate into chondrocytes; adipogenic differentiation medium induces eMSCs to differentiate into adipocytes.

The surface markers of the PEMSCs involved in this invention are identified by flow cytometry. The antibodies suitable for identifying PEMSCs: CD44 (Abcam, ab95138), CD34 (Abcam, ab223930), CD105 (Abcam, ab53321), and CD11b (Abcam, ab34444).

The PEMSCs involved in the present invention are applied to the repair of cells such as intrauterine adhesions, skin and vascular injury, including the repair of premature ovarian failure and endometrial injury, the repair method is simple and easy, and it can be regenerated and repaired by implanting it into the lesion site with biomaterials.

Compared with the existing technology, the present invention is isolated from the uterine tissue of large animals: pigs, the steps are simple and the amount of cells is large, and the cultured EMSCs have phenotypes of CD34−, CD11b−, CD44+, and CD105+. They can be induced to differentiate into osteoblasts, adipocytes, and chondrocytes in vitro, and they can repair various tissue and organ injuries. This invention provides a new pathway for cell transplantation therapy. By isolating MSCs from porcine endometrial tissue and culturing and expanding them in a culture medium containing cell growth factors, these cells can be induced to differentiate into multiple cell types and have broad application prospects in tissue engineering and regenerative medicine.

### BRIEF DESCRIPTION OF DRAWINGS

FIG. 1 is a schematic diagram of the growth state of cells of the P0 generation after 5 days of culture in a CO₂ incubator according to the present invention.

FIG. 2 is a schematic diagram of the growth state of cells of the P2 generation after 3 days of culture in a CO₂ incubator according to the present invention, wherein, FIG. 2A shows 40× and FIG. 2B shows 100×.

FIG. 3 is a schematic diagram of the result of the flow cytometry examination of cell surface specific markers (CD44) according to the present invention.

FIG. 4 is a schematic diagram of the result of the flow cytometry examination of cell surface specific markers (CD11b) according to the present invention.

FIG. 5 is a schematic diagram of the result of the flow cytometry examination of cell surface specific markers (CD105) according to the present invention.

FIG. 6 is a schematic diagram of the result of the flow cytometry examination of cell surface specific markers (CD34) according to the present invention.

FIG. 7 is a schematic diagram of osteogenic induction result, wherein, FIG. 7A is the morphology of cells in PEMSCs after 21 days of osteogenic induction of a control group and FIG. 7B is the morphology of numerous bone nodules after osteoinduction of alizarin red stained PEMSCs according to the present invention.

FIG. 8 is a schematic diagram of of adipogenic induction result, wherein, FIG. 8A is the morphology of cells in PEMSCs after 26 days of adipogenic induction of a control group and FIG. 8B is the morphology of oil red O staining showed that the cells contained a large number of fat particles according to the present invention.

FIG. 9 is a schematic diagram of the morphology of the cartilage ball after the immunohistochemical experiment after 24 days of chondrogenic induction, wherein, FIG. 9A is 50 μm according to the present invention and FIG. 9B is 25 μm according to the present invention.

### DETAILED DESCRIPTION OF THE INVENTION

The following detailed explanations are provided in conjunction with the figures and specific implementation methods to further illustrate the present invention.

### Embodiment 1

The specific process flow of an isolation and culture method for PEMSCs involved in this embodiment is as follows:
(1) Fresh porcine uterus (with ovaries) tissue obtained from a local slaughterhouse is immediately placed in cold PBS and transported to a sterile laboratory within 2 hours;
(2) The tissue is first washed with sterile saline to remove surface blood stains, sterilized, and then soaked in sterile PBS containing Ca²⁺ and Mg²⁺ (pH 7.4) for 6 hours, followed by two rinses;
(3) The tissue is cut into small tissue blocks of 5 cm² using scissors that have been sterilized in an autoclave, and then soaked again in sterile PBS containing Ca²⁺ and Mg²⁺ (pH 7.4) for 6 hours, followed by two rinses;
(4) The small tissue blocks are cut open in the middle to expose the endometrial layer. The serosal layer is removed using forceps, and the tissue containing the endometrial and myometrial layers is obtained. The myometrial layer is carefully and slowly removed from the endometrial layer using forceps and scissors. The endometrial layer is then cut into 1 mm² pieces and placed into a basic MSCCM supplemented with 0.2% type II collagenase and 40 μg/mL DNase I. The mixture is digested at 37° C. in a culture incubator at 150 rpm for 2-3 hours, with inversion mixing every 15 minutes to obtain a cell suspension.
(5) the cell suspension was filtered through 100 and 40 micron cell filters, digestion was further prevented by the addition of MSCCM, and centrifuged at 1000 rpm for 5 minutes, secondly, the cells were treated with red blood cell lysate twice and washed with PBS twice, the cells were inoculated in MSCCM at a clonal density of 500 cell/cm², and incubated at 37.0° C. in a humidified atmosphere of 5% CO₂, on the second day after inoculation, the adherent status of the cells was observed closely, the fresh MSCCM was replaced every 2-3 days;
(6) When the cells reach 85% confluence, 1 mL of 0.25% trypsin is added for digestion for 2 minutes. When cell retraction and dispersion are observed under a microscope, an equal volume of complete culture medium is immediately added to stop the digestion. The cells are transferred to an EP tube and centrifuged at 1500 rpm for 5 minutes.

The supernatant is removed, and the cells are resuspended in complete culture medium. One-third of the cells are transferred to a new culture dish and gently mixed.

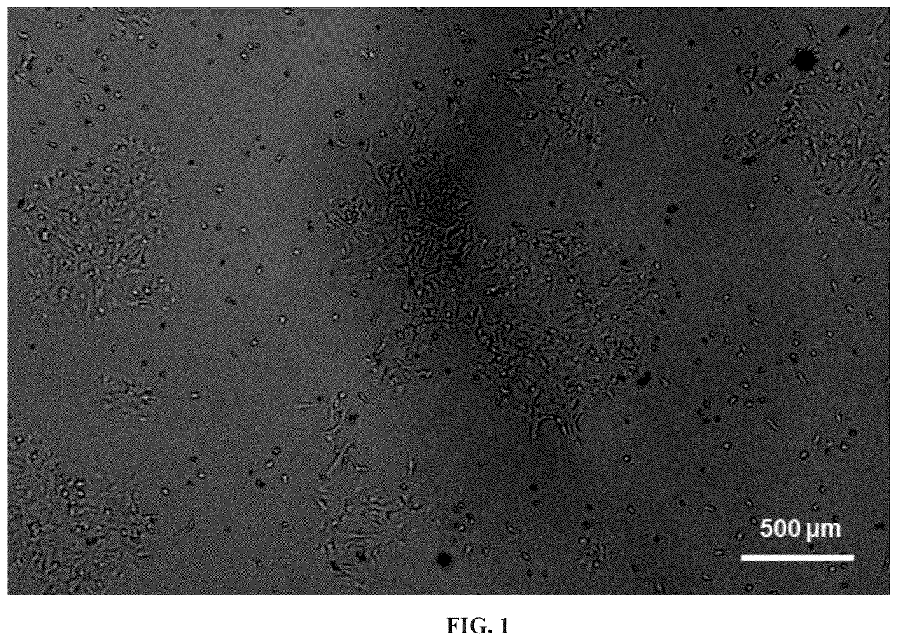

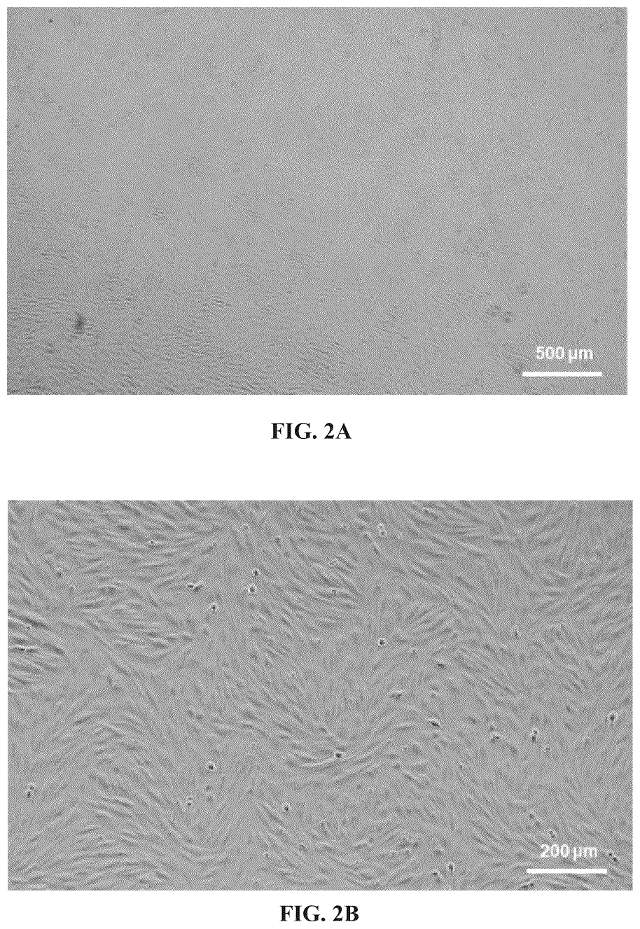

The cells were cultured in a CO₂ incubator, and after 24 hours, the cell state was observed under an inverted microscope, if bacterial contamination was observed, the cells were discarded, and the culture was continued without pollution. It was found that a single cell adhered to the wall, and the position of a single cell in the culture dish was marked for continuous observation; after 2-4 days, part of the original MSCCM was aspirated and supplemented with the fresh MSCCM; After 5-7 days of continuous culture, it was observed that the clone gradually extended to the surrounding, the clone gradually became larger, and the cell state gradually relaxed; after the cells reached 80-90% confluence, the cells were digested with trypsin and centrifuged, the P1 generation cells were obtained and transferred to another new culture dish after resuspension; the P5 generation cells cultured in this way still maintained strong proliferation ability; wherein the P0 generation cells were clone-like growth morphology as shown in FIG. 1, and the P2 generation cells were vigorous proliferation and long spindle growth morphology as shown in FIG. 2, which was similar to fibroblasts.

### Embodiment 2

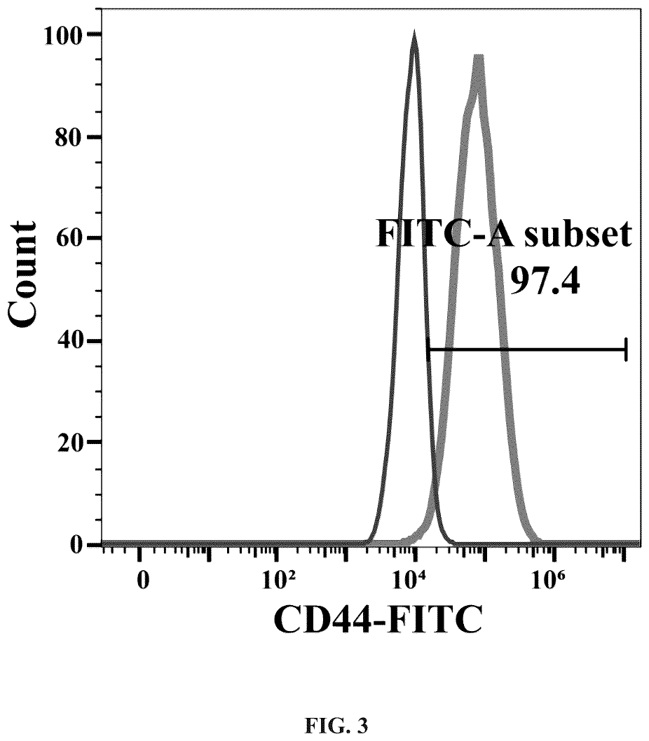

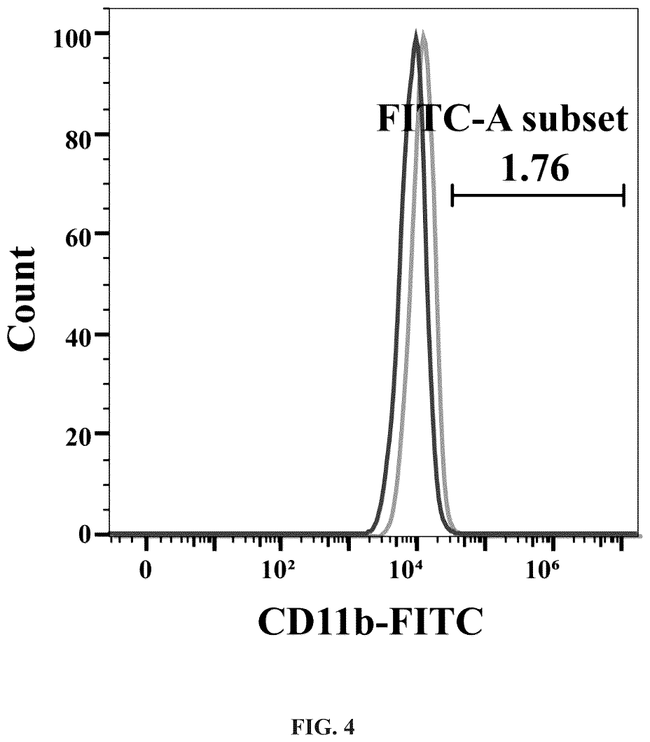

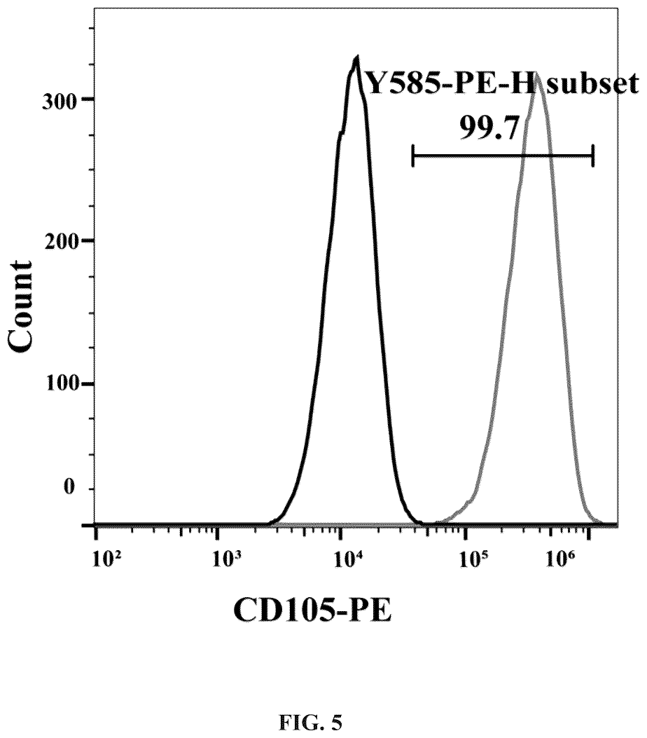

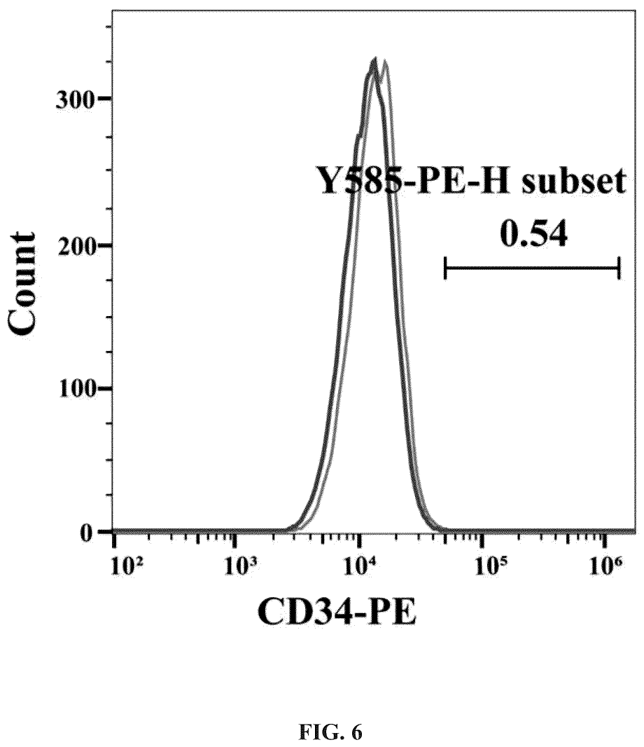

The process flow of phenotypic identification of PEMSCs involved in this embodiment is as follows:
(1) The cells of P2-P3 generation were inoculated in 6 cm² culture dish;
(2) When the cell density reaches 60-80%, 1 mL of 0.25% trypsin is added for digestion for 2 minutes, followed by centrifugation at 1500 rpm for 5 minutes.
(3) The cells are washed once with PBS and twice with cell staining buffer containing BSA (bovine serum albumin).
(4) Five EP tubes are prepared, with one tube serving as a blank control. FITC (fluorescein isothiocyanate)-labeled CD44 and CD11b, and PE (phycoerythrin)-labeled CD34 and CD105 are added to the other four EP tubes, respectively, at 2 μL per tube. The cells are incubated at 4° C. in the dark on ice for 30 minutes.
(5) After washing twice with cell staining buffer, the cells are resuspended in 200 μL of cell staining buffer and analyzed using a flow cytometer.
(6) the results are shown in FIGS. 3-6, after analysis, it was found that the subculture PEMSCs all expressed CD44 (97.4%) and CD105 (99.7%), and the expression rate was greater than 95%, which accords with the international standard of mesenchymal stem cell phenotype;
They do not express CD34 (0.54%) and CD11b (1.76%), with expression rates less than 2%, which also meets the international standards for mesenchymal stem cell phenotypes.

This demonstrates that the isolated and cultured porcine endometrial cells fully meet the characteristics of MSCs and are identified as PEMSCs.

### Embodiment 3

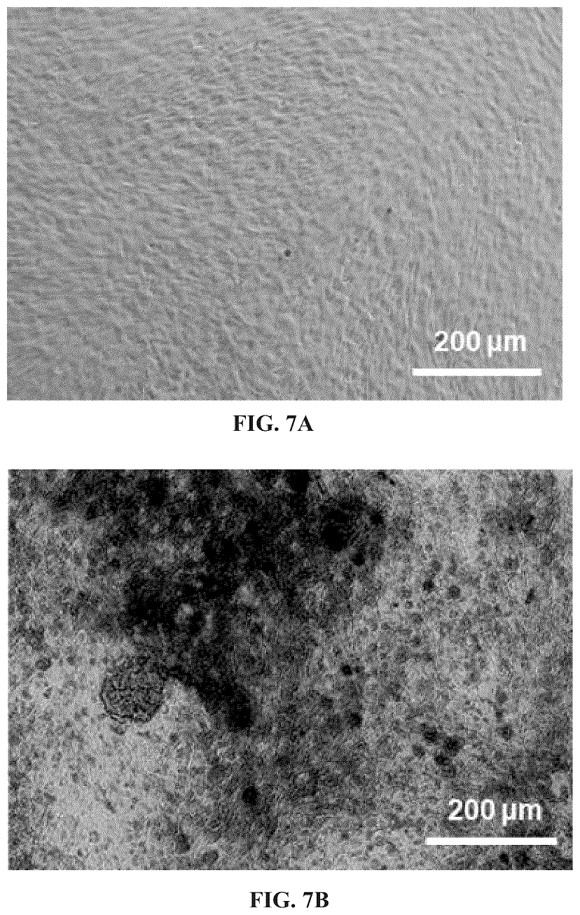

The process for differentiating PEMSCs into osteoblasts in this example is as follows:
(1) PEMSCs obtained from embodiment 1 were cultured to P3;
(2) PEMSCs were induced according to the OriCell® adipose-derived MSCs osteogenic induced differentiation kit (GUXMD-90021). After 24 days of induction, the cells are gently stained with Alizarin Red working solution and observed under a microscope for osteogenic staining effects. The results are shown in FIG. 7. Compared with the MSCs before induction, the induced cells exhibit a large number of calcium nodules after staining with alizarin red working solution, indicating that p PEMSCs can differentiate into osteoblasts.

### Embodiment 4

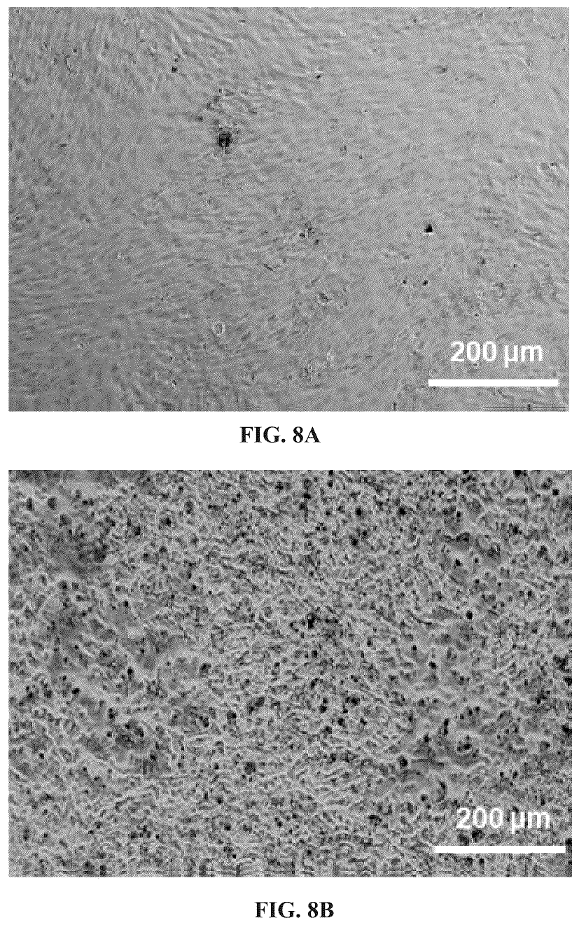

The process for differentiating PEMSCs into adipocytes in this example is as follows:
(1) PEMSCs obtained from embodiment 1 were cultured to P3;
(2) PEMSCs were induced according to the OriCell® adipose-derived MSCs osteogenic induced differentiation kit (GUXMD-90031), after 26 days of induction culture, oil red O dye operating solution was used for gentle staining, the culture plate was placed under a microscope to observe the effect of adipogenic staining, the results were shown in FIG. 8, compared with the MSCs before induction, a large number of lipid droplets appeared in the induced cells after staining with oil red O dye operating solution, indicating that PEMSCs can differentiate into adipocytes.

After 26 days of induction, the cells are gently stained with oil red O working solution and observed under a microscope for adipogenic staining effects. The results are shown in FIG. 8. Compared with the MSCs before induction, the induced cells showed a large number of lipid droplets after staining with oil red O working solution, indicating that PEMSCs can differentiate into adipocytes.

### Embodiment 5

The process for differentiating PEMSCs into chondrocytes in this example is as follows:
(1) PEMSCs obtained from embodiment 1 were cultured to P3;
(2) PEMSCs were induced according to the OriCell® adipose-derived MSCs osteogenic induced differentiation kit (GUXMD-90041),

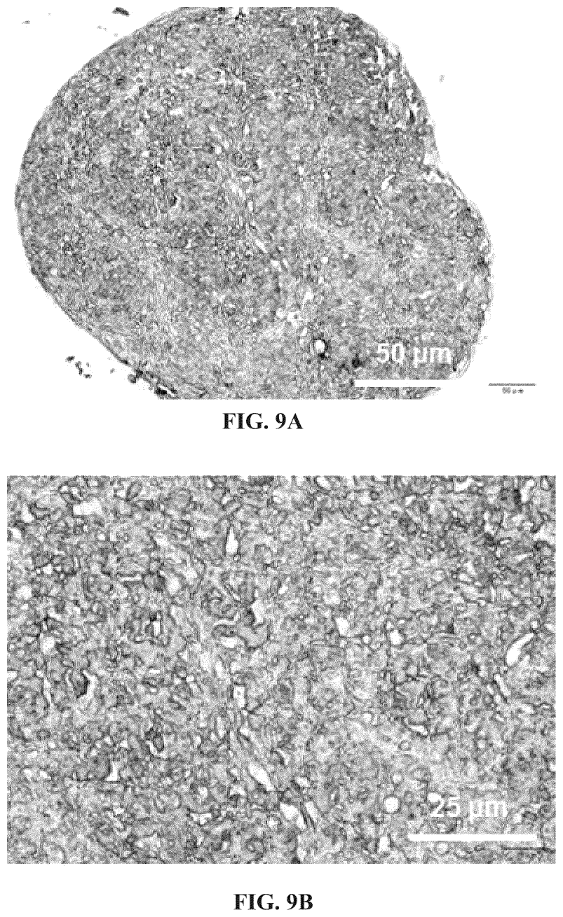

After 24 days of induction, the chondrogenic pellets are embedded and sectioned, and then stained with alcian blue working solution. The staining effects are observed using a panoramic scanner. The results are shown in FIG. 9. Most of the induced PEMSCs were stained with alcian blue, indicating that they can differentiate into chondrocytes.

What is claimed is:

1. An isolation and culture method for porcine endometrial mesenchymal stem cell (EMSCs), wherein the specific process comprises the following steps:
(1) Pre-treatment
Firstly, in the sterile room, cutting the porcine uterus, using pre-cooled sterile phosphate-buffered saline (PBS) added with magnesium chloride and calcium chloride to immerse, and rinsing;
Secondly, peeling a serosa layer off to separate the endometrial layer from the muscular layer;
Finally, placing the fragmented intima layer in an incubator at 37° C. to be digested with a basal medium supplemented with a digestive enzyme for 2-3 hours, and blowing every 15 minutes to obtain a cell suspension;
(2) Centrifugation
Firstly, filtering the cell suspension through cell filters, further preventing digestion by the addition of the MSCs complete culture medium (MSCCM), washing with PBS, and centrifuging at 1000 rpm for 5 minutes;
Secondly, treating the cells with red blood cell lysate twice and washing with PBS;
Finally, inoculating the cells in the MSCCM, and incubating at 37.0° C. in a humidified atmosphere of 5% CO₂, replacing the MSCCM every 2-3 days, and the cells are P0 generation cells;
(3) Cultivation and passage
Firstly, pre-heating the MSCCM, trypsin with a concentration of 0.25% and PBS in a water bath at 37° C. in advance, when the cell density reaches 60%-80%, aspirating the MSCCM, and washing with PBS;
Secondly, adding trypsin for digestion, when the cells are shrunk and dispersed, adding immediately an equal amount of MSCCM to terminate the digestion, transferring the cells to an EP tube and centrifuging at 1000 rpm for 5 minutes;
Finally, removing the supernatant, adding MSCCM to re-suspend the cells, taking ⅓ of them and adding them into a new culture dish, shaking gently and mixing well, placing in a CO₂ incubator for cultivation, and the obtained cells are P1 generation cells;
(4) Counting, viability detection and culture
Taking 100 μL of P1 generation cells, detecting the cell activity and counting, adjusting the cell density, inoculating the cells into the MSCCM for cultivation, and the cells after subculture again being the P2 generation cells, and by analogy.

2. The isolation and culture method for PEMSCs according to claim 1, wherein the P2-P3 generation cells are induced to differentiate into osteoblasts, chondrocytes, adipocytes and so on by adding different cell differentiation mediums.

3. The isolation and culture method for PEMSCs according to claim 1, wherein in step (1), the concentration of magnesium chloride and calcium chloride added in sterile PBS is 1 mM and 0.5 mM, respectively; digestive enzymes contain 0.2% collagenase II and 40 μg/mL deoxyribonuclease I.

4. An isolation and culture method and application for PEMSCs according to claim 1, wherein in steps (2) and (3), the complete medium of MSC is MSC basal culture medium (MSCBM) containing 10% fetal bovine serum (FBS) and 1% DA.

5. The isolation and culture method and application for PEMSCs according to claim 1, wherein in step (3), the culture conditions are: a temperature of 37.0° C., and 5 % CO₂ saturation.

6. The isolation and culture method for PEMSCs according to claim 1, wherein in step (4), the inoculation concentration of the subculture cells is 0.5×10⁴/cm²-1.5×10⁴/cm².

7. The isolation and culture method and application for PEMSCs according to claim 2, wherein cells are induced to differentiate into three-line induced differentiation of MSCs comprising osteogenic differentiation, chondrogenic differentiation and adipogenic differentiation.

8. The isolation and culture method and application for PEMSCs according to claim 4, wherein the double antibiotics is 100 U/mL penicillin and 100 μg/mL streptomycin.

9. The isolation and culture method for PEMSCs according to claim 1, wherein through the identification of surface markers, the antibodies suitable for the identification of PEMSCs are determined: CD44, CD34, CD105, and CD11b.

10. An application for PEMSCs, wherein the porcine endometrial mesenchymal stem cell is applied to repair intrauterine adhesions, skin and vascular injury cells, comprising premature ovarian failure, and endometrial injury.

11. The isolation and culture method for PEMSCs according to claim 3, wherein through the identification of surface markers, the antibodies suitable for the identification of PEMSCs are determined: CD44, CD34, CD105, and CD11b.

12. The isolation and culture method for PEMSCs according to claim 4, wherein through the identification of surface markers, the antibodies suitable for the identification of PEMSCs are determined: CD44, CD34, CD105, and CD11b.

13. The isolation and culture method for PEMSCs according to claim 5, wherein through the identification of surface markers, the antibodies suitable for the identification of PEMSCs are determined: CD44, CD34, CD105, and CD11b.

14. The isolation and culture method for PEMSCs according to claim 6, wherein through the identification of surface markers, the antibodies suitable for the identification of PEMSCs are determined: CD44, CD34, CD105, and CD11b.

15. The isolation and culture method for PEMSCs according to claim 8, wherein through the identification of surface markers, the antibodies suitable for the identification of PEMSCs are determined: CD44, CD34, CD105, and CD11b.
